(19) United States
(12) Patent Application Publication
(10) Pub. No.: US 2026/0000667 A1
(43) Pub. Date: Jan. 1, 2026

(54) STABLE PHARMACEUTICAL COMPOSITION OF BUPRENORPHINE AND PREPARATION METHOD AND USE THEREOF

(71) Applicant: ALAR PHARMACEUTICALS INC., Taiwan (CN)

(72) Inventors: Tong-Ho LIN, Taiwan (CN); Yung-Shun WEN, Taiwan (CN); Chia-Hsien CHEN, Taiwan (CN)

(21) Appl. No.: 18/878,327

(22) PCT Filed: Jun. 21, 2023
(86) PCT No.: PCT/CN2023/101708

§ 371 Date: Dec. 23, 2024

Related U.S. Application Data
(60) Provisional application No. 63/355,421, filed on Jun. 24, 2022.

(51) Int. Cl. A61K 31/485; A61K 9/08; A61K 47/10; A61K 47/18; A61K 47/22

(52) U.S. Cl. CPC A61K 31/485; A61K 9/08; A61K 47/10; A61K 47/18; A61K 47/22

(57) ABSTRACT

Provided is a pharmaceutical composition including buprenorphine, a derivative thereof, or a pharmaceutically acceptable salt thereof, a stabilizer, a biocompatible solvent, and at least one compound represented by Formula (IIA), (IIB) or (IIC), wherein R₁ is hydrogen, a C₁-C₂₀ alkylcarbonyl group, or a C₆-C₁₈ arylcarbonyl group, and wherein the compound of Formula (IIA), (IIB) and (IIC) each have an amount of less than 1% w/w based on the total weight of the pharmaceutical composition during a shelf life of the pharmaceutical composition. Also provided is a method for preparing the pharmaceutical composition and a method for treating opioid addiction, pain, and/or depression in a subject in need thereof by administering the pharmaceutical composition to the subject.

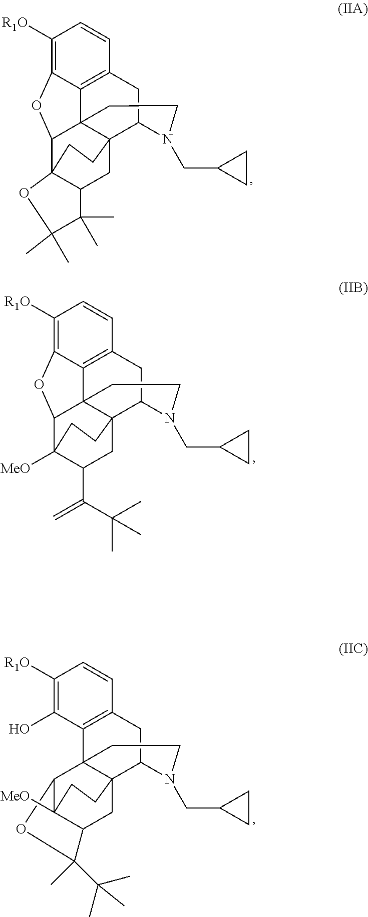

### TECHNICAL FIELD

The present disclosure relates to a stable formulation of buprenorphine, particularly to a pharmaceutical composition for the treatment of opioid dependence, pain, and depression, comprising buprenorphine or a derivative or a pharmaceutically acceptable salt thereof, a biocompatible solvent, and a stabilizer.

### BACKGROUND

Buprenorphine is indicated for the treatment of pain and opioid dependence in several dosage forms, e.g., BUVIDAL (subcutaneous injection), BUPRENEX (intravenous or intramuscular injection), BUTRANS (transdermal film), SUBLOCADE (subcutaneous injection), SUBOXONE (sublingual tablet or sublingual film), and SUBUTEX (sublingual tablet).

It is well-known in the art that buprenorphine would degrade to produce impurities through oxidation, dehydration, ring opening, rearrangement pathway, and the like. DenHerder et al. (J. Am. Assoc. Lab. Anim. Sci. (2017) 56(4): 457-461) disclosed a chemical stability test of the buprenorphine injection diluted with saline, indicating that the concentration of buprenorphine stored in glass vials was significantly decreased, with 95.7% remaining when refrigerated after 30 days and 94.2% remaining at room temperature after 60 days. The decrease of buprenorphine may result from degradation. Therefore, it would be necessary to develop a buprenorphine composition having better stability.

In previous studies, one method to enhance the stability of buprenorphine was proposed by an antioxidation pathway. However, buprenorphine could be degraded to impurities by a non-oxidation pathway. For example, Hotha et al. (Am. J. Analyt. Chem. (2016) 7, 107-140) disclosed a buprenorphine degradation pathway and indicated that according to European Pharmacopoeia (Ph. Eur.) monograph, buprenorphine was degraded to Impurity I due to the dehydrative cyclization of buprenorphine to form a furanyl ring.

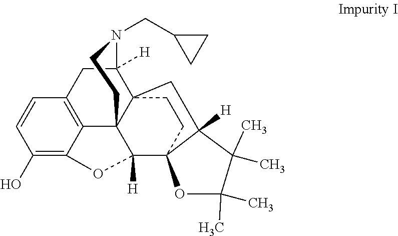

According to Ph. Eur. monograph, there are 10 different impurities of buprenorphine hydrochloride, and related substances are monitored. For example, Impurities A, B, F, and J are limited to be not more than 0.2%; Impurity G is limited to be not more than 0.15%; and Impurity H is limited to be not more than 0.25%. Among these impurities, Impurities A, B, F, G, H, and J are formed from non-oxidation pathways. Similarly, according to British pharmacopoeia, it is reported that a buprenorphine formulation would generate Impurities A, B, F, and G, and excessive impurities may cause unknown health hazard to human.

In addition, the appearance of a conventional pharmaceutical composition containing buprenorphine may show discoloration during the shelf life, which significantly influences product acceptability and commercial value of buprenorphine in the market.

Therefore, there is still an unmet and urgent need in the art to provide a stable pharmaceutical composition of buprenorphine or a derivative or a pharmaceutically acceptable salt thereof, which contains decreased impurities formed by the non-oxidation pathway and prevents discoloration during a shelf life at room temperature.

### SUMMARY

In view of the foregoing, the present disclosure provides a stable pharmaceutical composition of buprenorphine, which comprises buprenorphine, a derivative thereof, and a pharmaceutically acceptable salt thereof as an active pharmaceutical ingredient and an impurity at a relatively low amount.

In at least one embodiment of the present disclosure, the pharmaceutical composition comprising an active pharmaceutical ingredient and at least one compound represented by Formula (IIA), (IIB) or (IIC) below:

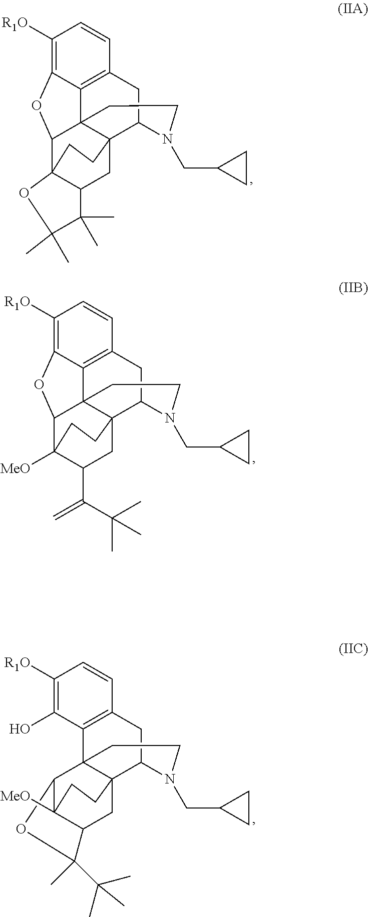

wherein R₁ is hydrogen, a C₁-C₂₀ alkylcarbonyl group, or a C₆-C₁₈ arylcarbonyl group.

In at least one embodiment of the present disclosure, the active pharmaceutical ingredient in the pharmaceutical composition is at least one of buprenorphine, a derivative thereof, and a pharmaceutically acceptable salt thereof. In some embodiments, the buprenorphine and the buprenorphine derivative may be represented by Formula (I) below:

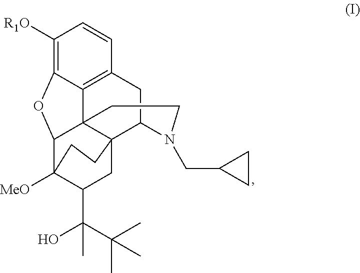

wherein R₁ is as defined above.

In at least one embodiment of the present disclosure, the active pharmaceutical ingredient in the pharmaceutical composition may be the buprenorphine derivative and/or the pharmaceutically acceptable salt of buprenorphine. In some embodiments, the active pharmaceutical ingredient in the pharmaceutical composition may be buprenorphine acetate, buprenorphine pentanoate, buprenorphine hexanoate, buprenorphine pivalate, buprenorphine decanoate, buprenorphine stearate, buprenorphine benzoate, or buprenorphine hydrochloride.

In at least one embodiment of the present disclosure, the active pharmaceutical ingredient in the pharmaceutical composition is present in an amount of 1% to 99% w/w, based on the total weight of the pharmaceutical composition. In some embodiments, an amount of the buprenorphine, the derivative thereof, and the pharmaceutically acceptable salt thereof in the pharmaceutical composition has a lower limit chosen from 5%, 10%, 15%, 20%, 25%, 30%, 35%, and 40% of the pharmaceutical composition by weight, and an upper limit chosen from 80%, 75%, 70%, 65%, 60%, 55%, 50%, and 45% of the pharmaceutical composition by weight. In some embodiments, the buprenorphine, the derivative thereof, and the pharmaceutically acceptable salt thereof as the active pharmaceutical ingredient may be present in an amount range of 5% to 80%, such as 5% to 80%, 5% to 70%, 5% to 60%, 10% to 80%, 10% to 70%, 10% to 60%, 20% to 80%, 20% to 70%, and 20% to 60%, based on the total weight of the pharmaceutical composition.

In at least one embodiment of the present disclosure, the compounds of Formula (IIA), (IIB) and (IIC) each in the pharmaceutical composition are present in an amount less than 1% w/w, based on the total weight of the pharmaceutical composition. For example, the compounds of Formula (IIA), (IIB) and (IIC) each have an amount of less than 1% w/w, less than 0.95% w/w, less than 0.9% w/w, less than 0.85% w/w, less than 0.8% w/w, less than 0.75% w/w, less than 0.7% w/w, less than 0.65% w/w, less than 0.6% w/w, less than 0.55% w/w, less than 0.5% w/w, less than 0.45% w/w, or less than 0.4% w/w.

In at least one embodiment of the present disclosure, the total amount of the compounds of Formula (IIA), (IIB) and (IIC) is less than 1% w/w, such as less than 0.9% w/w, less than 0.8% w/w, less than 0.7% w/w, less than 0.6% w/w, or less than 0.5% w/w, during a shelf life of the pharmaceutical composition. In some embodiments, the amounts of each of the compounds of Formula (IIA), (IIB) and (IIC) are not 0% in the pharmaceutical composition at the same time. For example, the amount of the compound of Formula (IIA), the amount of the compound of Formula (IIB), and the amount of the compound of Formula (IIC) are not 0% in the pharmaceutical composition at the same time.

In at least one embodiment of the present disclosure, when the compounds of Formula (IIA), (IIB) or (IIC) is present in the pharmaceutical composition during a shelf life of the pharmaceutical composition, the compounds each have an amount of 0% to 1%, such as less than 0.9% w/w, less than 0.8% w/w, less than 0.7% w/w, less than 0.6% w/w, less than 0.5% w/w, less than 0.4% w/w, less than 0.3% w/w, less than 0.2% w/w, or less than 0.1% w/w, based on the total weight of the pharmaceutical composition. In some embodiments, the compounds of Formula (IIA), (IIB) and (IIC) each are present in an amount of 0% to 1% w/w during a shelf life of the pharmaceutical composition.

In at least one embodiment of the present disclosure, the shelf life of the pharmaceutical composition is at least 1 month, such as 2 months, 3 months, 4 months, 5 months, 6 months, 7 months, 8 months, 9 months, 10 months, 11 months, 12 months, 18 months, 20 months, 24 months, 30 months, 36 months, 40 months, 42 months, 4 years, 4.5 years, and 5 years at an ambient temperature or a temperature below the ambient temperature. In some embodiments, the ambient temperature may be in a range of temperatures, e.g., 20° C. to 25° C., 22° C. to 28° C., and 20° C. to 28° C. In yet some embodiments, the temperature below the ambient temperature may be a temperature lower than 28° C., such as 2° C. to 8° C., 22° C. to 25° C., 20° C. to 22° C., 2° C. to 25° C., 2° C. to 15° C., 2° C. to 10° C., 1° C. to 20° C., or 1° C. to 10° C.

In at least one embodiment of the present disclosure, the pharmaceutical composition may further comprise a stabilizer. In some embodiments, the stabilizer is selected from the group consisting of vitamin E (VitE) or a derivative thereof, ascorbic acid (VitC), butylated hydroxyanisole (BHA), and a combination thereof. In some embodiments, the vitamin E or the derivative thereof may be α-tocopherol, β-tocopherol, γ-tocopherol, δ-tocopherol, α-tocotrienol, β-tocotrienol, γ-tocotrienol, δ-tocotrienol, or D-α-tocopherol polyethylene glycol 1000 succinate (TPGS).

In at least one embodiment of the present disclosure, the stabilizer in the pharmaceutical composition is present in an amount of 0.01% w/w to 10% w/w, based on the total weight of the pharmaceutical composition. For example, the amount of the stabilizer may be 0.05% to 5%, 0.1% to 5%, 0.2% to 5%, 0.5% to 5%, 0.05% to 3%, 0.1% to 3%, 0.2% to 3%, 0.5% to 3%, 0.05% to 2%, 0.1% to 2%, 0.2% to 2%, 0.5% to 2%, 0.05% to 1%, 0.1% to 1%, 0.2% to 1%, or 0.5% to 1% of the pharmaceutical composition by weight.

In at least one embodiment of the present disclosure, the pharmaceutical composition may further comprise a biocompatible solvent. In some embodiments, the biocompatible solvent is selected from the group consisting of water, N-methyl-2-pyrrolidone (NMP), polyethylene glycol 400 (PEG400), polyethylene glycol 4000 (PEG4000), ethyl acetate, ethanol, butanol, 2-butanol, isobutanol, isopropanol, glycerin, benzyl benzoate (BnBzO), dimethyl sulfoxide, N,N-dimethylacetamide (DMA), propylene glycol, dimethyl glycol, benzyl alcohol, and any combination thereof.

In at least one embodiment of the present disclosure, the biocompatible solvent in the pharmaceutical composition is present in an amount of 20% w/w to 95% w/w, based on the total weight of the pharmaceutical composition. For example, the amount of the biocompatible solvent may be 20% to 95%, 20% to 90%, 20% to 80%, 20% to 70%, 20% to 60%, 20% to 50%, 30% to 95%, 30% to 90%, 30% to 80%, 30% to 70%, 30% to 60%, or 30% to 50% of the pharmaceutical composition by weight.

In at least one embodiment, the present disclosure also provides a method for preparing the above-mentioned stable pharmaceutical composition. The method for preparing the pharmaceutical composition comprises adding an active pharmaceutical ingredient and a stabilizer in a biocompatible solvent to form the pharmaceutical composition, wherein the active pharmaceutical ingredient is at least one of buprenorphine, a derivative thereof, and a pharmaceutically acceptable salt thereof.

In at least one embodiment, the present disclosure also provides a method for treating opioid addiction, pain, and/or depression in a subject in need thereof, comprising administering to the subject with a therapeutically effective amount of the above-mentioned pharmaceutical composition.

In some embodiments, the stable pharmaceutical composition of the present disclosure comprising a buprenorphine, a derivative thereof, or a pharmaceutically acceptable salt thereof as an active pharmaceutical ingredient and a stabilizer may reduce the impurities formed by the active pharmaceutical ingredient, such as those formed by a non-oxidation pathway. Therefore, the pharmaceutical composition of the present disclosure may improve the stability of the active pharmaceutical ingredient and thus exhibit longer shelf life as well as prevent the pharmaceutical composition from discoloration.

### BRIEF DESCRIPTION OF THE DRAWINGS

The present disclosure can be more fully understood by reading the following descriptions of the embodiments, with reference made to the accompanying drawings.

FIG. 1 shows various degradation pathways of buprenorphine-based compounds and the structures of impurities formed from different degradation pathways.

FIG. 2 shows the profiles of Impurity A1 formation from Formulations F28 and F29, which are stored at 25° C., in accordance with the embodiments of the present disclosure. USL: upper specification limit.

FIG. 3 shows the profiles of Impurity A1 formation from Formulations F28 and F29, which are stored at 40° C., in accordance with the embodiments of the present disclosure. USL: upper specification limit.

FIG. 4 shows the appearance of Formulations F28 and F29, which are stored at 25° C., in accordance with the embodiments of the present disclosure. M: month(s).

FIG. 5 shows appearances of Formulations F28 and F29, which are stored at 40° C., in accordance with embodiments of the present disclosure. M: month(s).

### DETAILED DESCRIPTION

The following examples are used to exemplify the present disclosure. A person of ordinary skill in the art can understand other advantages of the present disclosure, based on the specification of the present disclosure. The present disclosure can also be implemented or applied as described in different examples. It is possible to modify and/or alter the following examples for carrying out this disclosure without contravening its scope for different aspects and applications.

It is noted that, as used in this disclosure, the singular forms “a,” “an,” and “the” include plural referents unless expressly and unequivocally limited to one referent. The term “or” is used interchangeably with the term “and/or” unless the context clearly indicates otherwise.

As used herein, the terms “comprising,” “including,” “having,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

As used herein, the terms “at least one” and “one or more” may have the same meaning and include one, two, three, or more.

Throughout this disclosure, ordinal numbers (e.g., first, second, third, etc.) may be used as an adjective for an element (i.e., any noun in the disclosure). The use of ordinal numbers is not to imply or create an ordering of the elements, nor to limit any element to be only a single element unless expressly indicated, such as by the use of the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

As used herein, the term “about” generally refers to the numerical value meant to encompass variations of +20%, +10%, +5%, +1%, +0.5%, or +0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by one of ordinary skill in the art. Unless otherwise expressly specified, all of the numerical ranges, amounts, values, and percentages such as those for quantities of materials, durations of time periods, temperatures, operating conditions, ratios of amounts, and the likes disclosed herein should be understood as modified in all instances by the term “about.”

The numeral ranges used herein are inclusive and combinable. Any numeral value that falls within the numeral scope herein could be taken as a maximum or minimum value to derive the sub-ranges therefrom. For example, it should be understood that the numeral range “1% to 99%” comprises any sub-ranges between the minimum value of 1% to the maximum value of 99%, such as the sub-ranges from 1% to 90%, from 20% to 80%, from 40% to 60%, and so on. In addition, a plurality of numeral values used herein can be optionally selected as maximum and minimum values to derive numerical ranges. For instance, the numerical ranges of 5% to 60%, 10% to 40%, and 20% to 60% can be derived from the numeral values of 5%, 10%, 20%, 40%, and 60%.

As used herein, the term “treat,” “treating,” or “treatment” encompasses partially or completely preventing, ameliorating, mitigating, and/or managing a symptom, a disorder, or a condition associated with a disease. The term “treat,” “treating,” or “treatment” as used herein refers to application or administration of one or more therapeutic agent or surgery to a subject, who has a symptom, a disorder, or a condition associated with a disease, with the purpose to partially or completely alleviate, ameliorate, relieve, delay onset of, inhibit progression of, reduce severity of, and/or reduce incidence of one or more symptoms, disorders, or conditions associated with the disease. Treatment may be administered to a subject who exhibits only an early sign of such symptoms, disorders, and/or conditions for the purpose of decreasing the risk of developing the symptoms, disorders, and/or conditions associated with a disease.

As used herein, the terms “subject,” “individual,” and “patient” may be interchangeable and refer to an animal, e.g., a mammal. The term “subject” is intended to refer to both the male and female gender unless one gender is specifically indicated. In at least one embodiment of the present disclosure, the subject is selected from the group consisting of a rodent, a murine, a monkey, a guinea pig, a dog, a cat, a cow, a sheep, a pig, a horse, a rabbit, and a human. In some embodiments, the subject is a human.

As used herein, the term “therapeutically effective amount” refers to the amount of an active agent or a composition that is sufficient to treat or prevent the specified disorder, condition, or disease in a subject in need thereof. Effective doses will vary, as recognized by one of ordinary skill in the art, depending on routes of administration, excipient usage, the possibility of co-usage with other treatment, and the condition to be treated.

The present disclosure is directed to a pharmaceutical composition comprising an active pharmaceutical ingredient, a stabilizer, a biocompatible solvent, and at least one compound represented by Formula (IIA), (IIB) or (IIC) below:

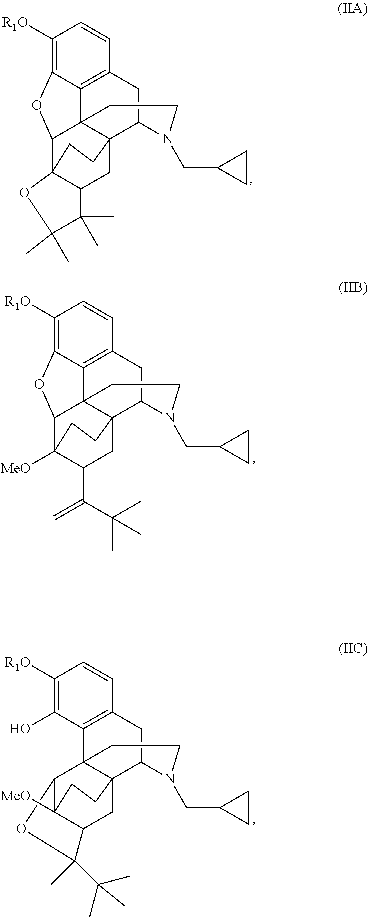

wherein R₁ is hydrogen, a C₁-C₂₀ alkylcarbonyl group, or a C₆-C₁₈ arylcarbonyl group.

As used herein, the term “impurity” refers to a chemical substance formed by the degradation of the active pharmaceutical ingredient. In at least one embodiment of the present disclosure, the active pharmaceutical ingredient is at least one of buprenorphine, a derivative thereof, and a pharmaceutically acceptable salt thereof, which may be degraded into impurities through oxidation or non-oxidation pathway (e.g., dehydration, ring opening, and rearrangement pathways). Accordingly, the impurity may refer to oxidation impurity, non-oxidation impurity, and individual unknown impurities.

As used herein, the compounds of Formula (IIA), (IIB) and (IIC) are also called Impurity A, Impurity B, and Impurity C, respectively, which refer to non-oxidation impurities degraded by the non-oxidation pathway of the active pharmaceutical ingredient. Accordingly, the structure of the compounds of Formula (IIA), (IIB) and (IIC) is correlated to the structure of the active pharmaceutical ingredient from which the compound is formed.

For example, the buprenorphine or the derivative thereof in the pharmaceutical composition may be represented by Formula (I) below:

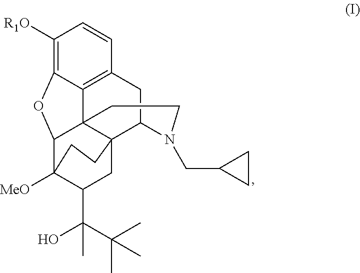

wherein R₁ is hydrogen, a C₁-C₂₀ alkylcarbonyl group, or a C₆-C₁₈ arylcarbonyl group, and the compounds of Formula (IIA), (IIB) and (IIC) may have the same R₁ group as the active pharmaceutical ingredient of Formula (I).

In at least one embodiment of the present disclosure, the compounds of Formula (IIA), (IIB) and (IIC) formed from buprenorphine hexanoate as the active pharmaceutical ingredient may be further represented by the following Impurity A1, Impurity B1, and Impurity C1, respectively:

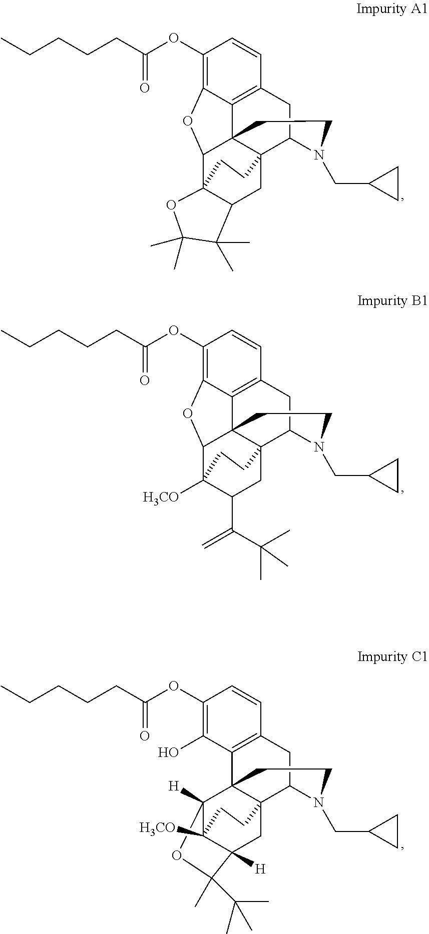

In at least one embodiment of the present disclosure, the compounds of Formula (IIA), (IIB) and (IIC) formed from buprenorphine decanoate as the active pharmaceutical ingredient may be further represented by the following Impurity A2, Impurity B2, and Impurity C2, respectively:

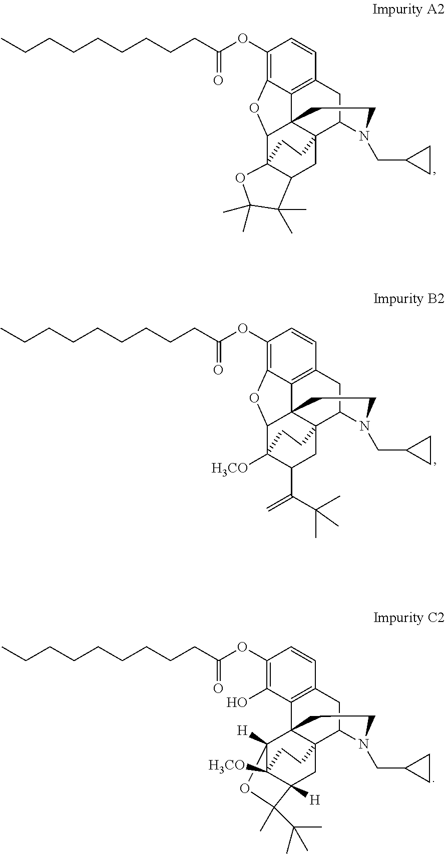

In at least one embodiment of the present disclosure, the compounds of Formula (IIA), (IIB) and (IIC) formed from buprenorphine acetate as the active pharmaceutical ingredient may be further represented by the following Impurity A3, Impurity B3, and Impurity C3, respectively:

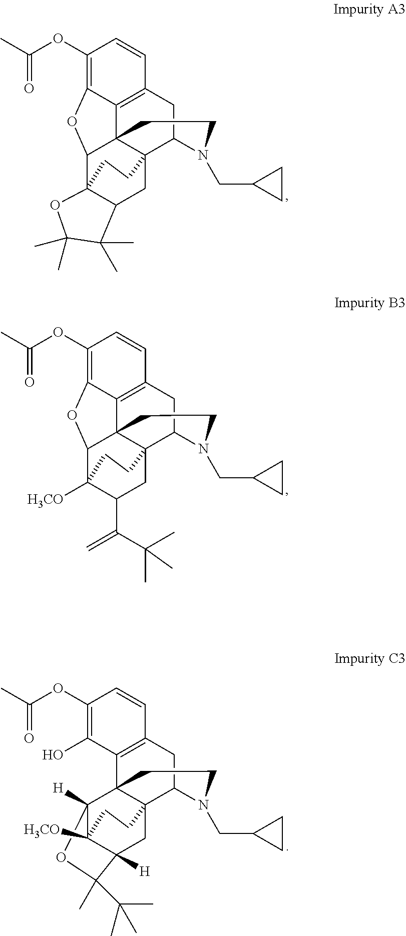

In at least one embodiment of the present disclosure, the total amount of the compounds of Formula (IIA), (IIB) and (IIC) in the pharmaceutical composition is in an amount of less than 1% based on the total weight of the pharmaceutical composition. In some embodiments, the compounds of Formula (IIA), (IIB) and (IIC) in the pharmaceutical composition are independently in an amount of 0% to 1% based on the total weight of the pharmaceutical composition. In some embodiments, the total impurities in the pharmaceutical composition may be in an amount of less than 1% based on the total weight of the pharmaceutical composition. In some embodiments, the impurities in the pharmaceutical composition has a total amount less than 1% w/w during the shelf life of the pharmaceutical composition.

In at least one embodiment of the present disclosure, the buprenorphine, the derivative thereof, or the pharmaceutically acceptable salt thereof is in an amount of 1% to 99% w/w based on the total weight of the pharmaceutical composition. In some embodiments, the buprenorphine, the derivative thereof, or the pharmaceutically acceptable salt thereof is in an amount of 5% to 80% w/w based on the total weight of the pharmaceutical composition. For example, the buprenorphine, the derivative thereof, or the pharmaceutically acceptable salt thereof may be in an amount of about 1% w/w, about 2% w/w, about 5% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, about 90% w/w, about 95% w/w, about 96% w/w, about 97% w/w, about 98% w/w, or about 99% w/w based on the total weight of the pharmaceutical composition, and not limited thereto.

In at least one embodiment of the present disclosure, the stabilizer is in an amount of 0.01% to 10% w/w based on the total weight of the pharmaceutical composition. In some embodiments, the stabilizer is in an amount of 0.1% to 2% w/w based on the total weight of the pharmaceutical composition. For instance, the stabilizer may be in an amount of about 0.01% w/w, about 0.02% w/w, about 0.04% w/w, about 0.06% w/w, about 0.08% w/w, about 0.1% w/w, about 0.2% w/w, about 0.5% w/w, about 1% w/w, about 1.5% w/w, about 2% w/w, about 4% w/w, about 6% w/w, about 8% w/w, or about 10% w/w, and not limited thereto.

In at least one embodiment of the present disclosure, the biocompatible solvent is in an amount of 20% to 95% w/w based on the total weight of the pharmaceutical composition. In some embodiments, the biocompatible solvent is in an amount of 30% to 95% w/w based on the total weight of the pharmaceutical composition, such as about 25%, about 30%, about 35%, about 38%, about 39% w/w, about 39.5% w/w, about 40% w/w, about 45% w/w, about 48% w/w, about 49% w/w, about 49.5% w/w, about 50% w/w, about 78% w/w, about 79% w/w, about 79.5% w/w, about 80% w/w, about 85% w/w, about 90% w/w, 94.5% w/w, or about 95% w/w, and not limited thereto.

In at least one embodiment of the present disclosure, a method for preparing the above pharmaceutical composition is provided. The method comprises adding an active pharmaceutical ingredient and a stabilizer in a biocompatible solvent to form a mixture of the pharmaceutical composition.

In at least one embodiment of the present disclosure, the method further comprises stirring the mixture to form a homogeneous solution. In some embodiments, the stirring may be performed for any time period, for example, about 5 min, about 10 min, about 15 min, about 20 min, or more, and not limited thereto. In some embodiments, the stirring may be performed under the ambient temperature, for example, about 0° C., about 5° C., about 10° C., about 15° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 50° C., about 55° C., about 60° C., about 65° C., about 70° C., about 75° C., or about 80° C., and not limited thereto.

In at least one embodiment of the present disclosure, the homogeneous solution is prepared under heating. In some embodiments, the mixture is heated to more than the ambient temperature to about 200° C., such as about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., or about 200° C., and not limited thereto. In some embodiments, the homogeneous solution may be prepared under 0° C. to 200° C., with or without heating.

In at least one embodiment of the present disclosure, the method further comprises filtering the homogeneous solution to obtain a filtered homogeneous solution. In some embodiments, the filtering is performed under a temperature in a range from the ambient temperature to about 200° C., such as about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., or about 200° C., and not limited thereto. For example, the filtering may be performed under a temperature in a range from 20° C. to 200° C., with or without heating.

In at least one embodiment of the present disclosure, the method further comprises sterilizing the homogeneous solution or the filtered homogeneous solution to obtain a sterilized pharmaceutical composition. In some embodiments, the sterilizing may be a thermal sterilization, a filtration sterilization, an irradiation sterilization, or any combination thereof. In yet some embodiments, the homogeneous solution may be sterilized under 100° C. to 200° C., such as about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., or about 200° C., and not limited thereto. In some embodiments, the homogeneous solution is sterilized for 10 to 120 minutes, such as about 10 min, about 15 min, about 20 min, about 25 min, about 30 min, about 35 min, about 40 min, about 45 min, about 50 min, about 55 min, about 60 min, about 65 min, about 70 min, about 75 min, about 80 min, about 85 min, about 90 min, about 95 min, about 100 min, about 105 min, about 110 min, about 115 min, or about 120 minutes, and not limited thereto. In some embodiments, when the sterilizing is performed by the filtration sterilization, the step of filtering the homogeneous solution may be performed together with the process of the filtration sterilization.

In at least one embodiment of the present disclosure, the active pharmaceutical ingredient may be buprenorphine free base, a buprenorphine-based compound, or a pharmaceutically acceptable salt thereof. In some embodiments, the buprenorphine-based compound is used interchangeably to refer to a buprenorphine derivative, which may be 3-acyl-buprenorphine. In some embodiments, the acyl group of 3-acyl-buprenorphine is an alkylcarbonyl group or an arylcarbonyl group. In yet some embodiments, an alkyl portion of the alkylcarbonyl group is a straight-chain alkyl or a branched-chain alkyl having 1 to 20 carbon atoms, and an aryl portion of the arylcarbonyl group is an aromatic ring having 6 to 18 carbon atoms.

In at least one embodiment of the present disclosure, a pharmaceutically acceptable salt of the 3-acyl-buprenorphine may be, for example, a salt of HCl, formate, acetate, or the like, and not limited thereto.

As used herein, the term “biocompatible solvent” refers to any solvents well-known in the art that is substantially non-toxic or unharmful to the living tissue or organism. For example, the biocompatible solvent of the present disclosure may be water, N-methyl-2-pyrrolidone (NMP), polyethylene glycol 400 (PEG400), polyethylene glycol 4000 (PEG4000), ethyl acetate, ethanol, butanol, 2-butanol, isobutanol, isopropanol, glycerin, benzyl benzoate (BnBzO), dimethyl sulfoxide, N,N-dimethylacetamide (DMA), propylene glycol, dimethyl glycol, benzyl alcohol, and any combination thereof, and not limited thereto.

In at least one embodiment of the present disclosure, the stabilizer is at least one selected from the group consisting of vitamin E (VitE) or a derivative thereof, ascorbic acid (VitC), butylated hydroxyanisole (BHA), and any combination thereof. In some embodiments, the vitamin E or the derivative thereof may be α-tocopherol, β-tocopherol, γ-tocopherol, δ-tocopherol, α-tocotrienol, β-tocotrienol, γ-tocotrienol, δ-tocotrienol, D-α-tocopherol polyethylene glycol 1000 succinate (TPGS), or any combination thereof.

In at least one embodiment of the present disclosure, the pharmaceutical composition comprises a reduced amount impurities formed by the degradation of the buprenorphine, the derivative thereof, or the pharmaceutically acceptable salt thereof via a non-oxidation or an oxidation pathway. In some embodiments of the present disclosure, the amount of impurities formed by the non-oxidation pathway is reduced.

In at least one embodiment of the present disclosure, the stabilizer prevents the pharmaceutical composition from discoloration. In some embodiments, the amount of the impurities formed by the degradation of the buprenorphine, the derivative thereof, or the pharmaceutically acceptable salt thereof are reduced by the presence of the stabilizer in the pharmaceutical composition. In some embodiments of the present disclosure, the amount of the impurities formed by the non-oxidation pathway is significantly reduced by the presence of the stabilizer in the pharmaceutical composition.

In at least one embodiment of the present disclosure, the pharmaceutical composition is formulated into a formulation for at least one selected from the group consisting of subcutaneous administration, intravenous administration, intramuscular administration, intradermal administration, transdermal administration, sublingual administration, topical administration, intraperitoneal administration, intraarticular administration, transmucosal administration, intraorgan administration, intraosseous administration, and oral administration.

The present disclosure further provides a method for treating opioid addiction, pain, and/or depression in a subject in need thereof, comprising administering a therapeutically effective amount of the pharmaceutical composition of the present disclosure.

In at least one embodiment of the method for treating opioid addiction, pain, and/or depression of the present disclosure, the administration is three times a day, twice a day, once per day, once every two days, once every three days, once every four days, once every five days, once every six days, once per week, twice per week, three times per week, four times per week, five times per week, six times per week, once every two weeks, once every three weeks, once every four weeks, once every five weeks, once every six weeks, once every eight weeks, once every ten weeks, once every twelve weeks, once every four months, once every five months, or once every six months. It should be appreciated that the period of the administration can be varied based on an actual need and is not limited to the above-mentioned period.

As used herein, the term “administering” or “administration” refers to the placement of an active ingredient into a subject by a method or route which results in at least partial localization of the active ingredient at a desired site to produce a desired effect. The active ingredient described herein may be administered by any appropriate route known in the art including, but not limited to, oral or parenteral routes.

In at least one embodiment of the method for treating opioid addiction, pain, and/or depression of the present disclosure, the pharmaceutical composition is administered to a subject in need thereof through subcutaneous administration, intravenous administration, intramuscular administration, intradermal administration, transdermal administration, sublingual administration, topical administration, intraperitoneal administration, intraarticular administration, transmucosal administration, intraorgan administration, intraosseous administration, or oral administration.

Discoloration of a pharmaceutical composition influences the product acceptability and commercial value in the market. The appearance of the conventional pharmaceutical composition of buprenorphine exhibits discoloration during the shelf life. For example, the color of SUBLOCADE changes from colorless to yellow and further to an amber solution through degradation, and BUVIDAL is originally a yellowish solution and discolored to a yellow solution. The acceptable appearance of a buprenorphine solution should be colorless, ivory, beige, vanilla, wheat, light yellow, corn silk yellow, or yellow, while the undesirable degradation would lead to dark yellow, green yellow, light orange, orange, dark orange, amber, bronze, chocolate, khaki, camel, olive, brown, or even black. Determining the color with the Gardner color scale through naked eye, SUBLOCADE has a color score of at least Gardner 8, and BUVIDAL has a color score of at least Gardner 6.

According to the manufacturer's instruction, SUBLOCADE should be stored at 2° C. to SoC with 18-month shelf life, and it should be discarded if left at room temperature for longer than 7 days. It thus can be seen that SUBLOCADE is restricted to a refrigerated storage condition, and its shelf life is relatively shorter than other products.

In at least one embodiment of the present disclosure, the pharmaceutical composition provided herein may maintain the apparent color thereof by adding a suitable stabilizer that can reduce the formation of impurities, e.g., non-oxidation impurities, from degradation of buprenorphine, a derivative thereof, or a pharmaceutically acceptable salt thereof. In some embodiments, the pharmaceutical composition has a color score of less than Gardner 5, such as less than Gardner 4, less than Gardner 3, or less than Gardner 2.

The present disclosure also provides a use of a pharmaceutical composition in the manufacture of a medicament for treating opioid addiction, pain, and/or depression, wherein the pharmaceutical composition includes buprenorphine, a derivative thereof, or a pharmaceutically acceptable salt thereof, a stabilizer, a biocompatible solvent, and an impurity at a low amount. The present disclosure further provides a pharmaceutical composition for use in the treatment of opioid addiction, pain, and/or depression, wherein the pharmaceutical composition includes buprenorphine, a derivative thereof, or a pharmaceutically acceptable salt thereof, a stabilizer, a biocompatible solvent, and an impurity at a low amount. The present disclosure further provides a pharmaceutical composition used in treating opioid addiction, pain, and/or depression, wherein the pharmaceutical composition includes buprenorphine, a derivative thereof, or a pharmaceutically acceptable salt thereof, a stabilizer, a biocompatible solvent, and an impurity at a low amount.

The following embodiments further demonstrate the efficacy of the present disclosure but should not be used to limit the scope of the present disclosure.

### Examples

The present disclosure is further described by means of the following examples. However, these examples are only illustrative of the disclosure and in no way limit the scope and meaning of the present disclosure. Many modifications and variations of the present disclosure will be apparent to those skilled in the art upon reading this specification, and can be made without departing from its scope.

Generally, the buprenorphine, the derivative thereof, or the pharmaceutically acceptable salt thereof contained in the formulations would be degraded into impurities, in which the degradation pathways include oxidation, dehydration, ring opening, and rearrangement pathways. In the stability test described herein, the major impurities of buprenorphine formulations are formed by the non-oxidation pathway. The examples are described as below.

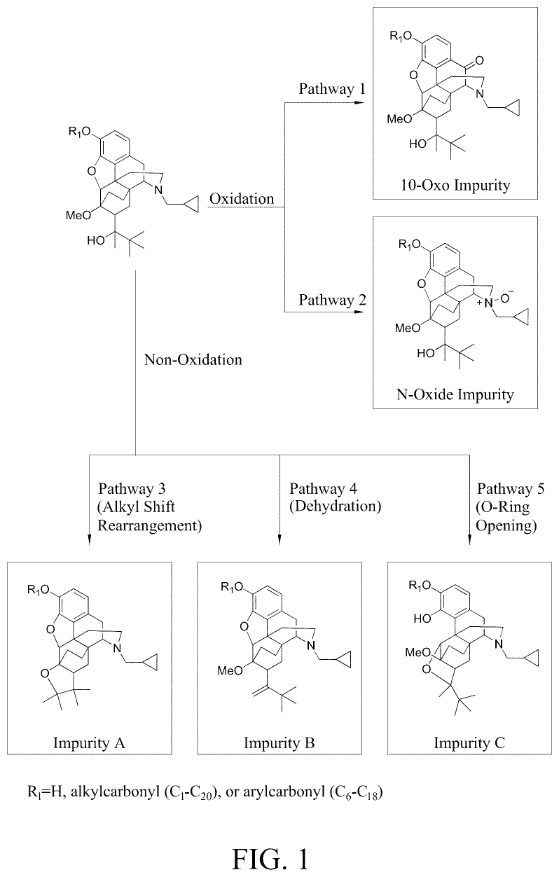

The characterization of Impurities A to C had been confirmed by nuclear magnetic resonance (NMR) spectroscopy. The structure of Impurities A to C were shown in FIG. 1, wherein R₁ represents H, alkylcarbonyl, or arylcarbonyl. In some embodiments, R₁ is alkylcarbonyl, and the alkyl portion may be a straight-chain alkyl or a branched-chain alkyl having 1 to 20 carbons. In some embodiments, R₁ is arylcarbonyl, and the aryl portion may be an aromatic ring having 6 to 18 carbons.

As shown in FIG. 1, Impurities A to C were formed by alkyl shift rearrangement, dehydration, and O-ring opening, respectively. The degradation pathway of Impurity A and Impurity B is the same as that of Impurity I and Impurity F of buprenorphine hydrochloride according to the Ph. Eur. monograph. Further, the 10-oxo impurity and N-oxide impurity of buprenorphine or the derivative thereof formed by an oxidation pathway were also shown in FIG. 1. However, the 10-oxo impurity and N-oxide impurity were not the major impurity in the stability test described herein.

### Example 1. Preparation of Buprenorphine Formulations

Buprenorphine, a derivative thereof, or a pharmaceutically acceptable salt thereof and a stabilizer were added into a glass vial containing a biocompatible solvent to form a mixture, wherein the buprenorphine, the derivative thereof, or the pharmaceutically acceptable salt thereof was used as an active pharmaceutical ingredient (API). Further, the mixture was subjected to sonication or stirring at ambient temperature until the API and the stabilizer were dissolved in the biocompatible solvent, thereby obtaining a homogenous solution. Afterward, the homogenous solution was sterilized by filtration sterilization at ambient temperature or by thermal sterilization at 121° C. for 15 minutes. Exemplary compositions, Formulations F01 to F40, were listed in Table 1 below, in which Formulations F01, F03, F05, F07, F15, F20, F23, F24, F29, F30, and F39 containing no stabilizers were used as comparative examples.

TABLE 1
Buprenorphine formulations prepared in Example 1

| Formulation | | Solvent, | Stabilizer, | |
|---|---|---|---|---|
| code | API, wt % | wt % | wt % | Sterilization |
| F01 | Buprenorphine free base, 20% | NMP, 80% | — | Filtration |
| F02 | Buprenorphine free base, 20% | NMP, 79.5% | VitE, 0.5% | Filtration |
| F03 | Buprenorphine free base, 20% | NMP, 80% | — | Thermal |
| F04 | Buprenorphine free base, 20% | NMP, 79.5% | VitE, 0.5% | Thermal |
| F05 | Buprenorphine acetate, 20% | NMP, 80% | — | Filtration |
| F06 | Buprenorphine acetate, 20% | NMP, 79.5% | VitE, 0.5% | Filtration |
| F07 | Buprenorphine hexanoate, | NMP, 80% | — | Filtration |
| | 20% | | | |
| F08 | Buprenorphine hexanoate, | NMP, 79.99% | VitE, 0.01% | Filtration |
| | 20% | | | |
| F09 | Buprenorphine hexanoate, | NMP, 79.9% | VitE, 0.1% | Filtration |
| | 20% | | | |
| F10 | Buprenorphine hexanoate, | NMP, 79.8% | VitE, 0.2% | Filtration |
| | 20% | | | |
| F11 | Buprenorphine hexanoate, | NMP, 79.5% | VitE, 0.5% | Filtration |
| | 20% | | | |
| F12 | Buprenorphine hexanoate, | NMP, 78% | VitE, 2% | Filtration |
| | 20% | | | |
| F13 | Buprenorphine hexanoate, 5% | NMP, 94.5% | VitE, 0.5% | Filtration |
| F14 | Buprenorphine hexanoate, | NMP, 49.5% | VitE, 0.5% | Filtration |
| | 50% | | | |
| F15 | Buprenorphine hexanoate, | NMP, 80% | — | Thermal |
| | 20% | | | |
| F16 | Buprenorphine hexanoate, | NMP, 79.5% | VitE, 0.5% | Thermal |
| | 20% | | | |
| F17 | Buprenorphine hexanoate, | NMP, 79.5% | VitC, 0.5% | Filtration |
| | 20% | | | |
| F18 | Buprenorphine hexanoate, | NMP, 79.5% | BHA, 0.5% | Filtration |
| | 20% | | | |
| F19 | Buprenorphine hexanoate, | NMP, 79.5% | TPGS, 0.5% | Filtration |
| | 20% | | | |
| F20 | Buprenorphine decanoate, | NMP, 80% | — | Filtration |
| | 20% | | | |
| F21 | Buprenorphine decanoate, | NMP, 79.5% | VitE, 0.5% | Filtration |
| | 20% | | | |
| F22 | Buprenorphine hexanoate, | NMP, 49.8% | VitE, 0.2% | Filtration |
| | 50% | | | |
| F23 | Buprenorphine hexanoate, 5% | NMP, 95% | — | Filtration |
| F24 | Buprenorphine hexanoate, | NMP, 50% | — | Filtration |
| | 50% | | | |
| F25 | Buprenorphine hexanoate, | NMP, 49.9% | VitC, 0.1% | Filtration |
| | 50% | | | |
| F26 | Buprenorphine hexanoate, | NMP, 49.8% | VitC, 0.2% | Filtration |
| | 50% | | | |
| F27 | Buprenorphine hexanoate, | NMP, 49.5% | VitC, 0.5% | Filtration |
| | 50% | | | |
| F28 | Buprenorphine hexanoate, | NMP, 49.8% | VitE, 0.2% | Thermal |
| | 50% | | | |
| F29 | Buprenorphine hexanoate, | NMP, 50% | — | Thermal |
| | 50% | | | |
| F30 | Buprenorphine decanoate, | NMP, 80% | — | Thermal |
| | 20% | | | |
| F31 | Buprenorphine decanoate, | NMP, 79.5% | VitE, 0.5% | Thermal |
| | 20% | | | |
| F32 | Buprenorphine hexanoate, | NMP, 79.9% | VitE, 0.1% | Thermal |
| | 20% | | | |
| F33 | Buprenorphine hexanoate, | NMP, 78% | VitE, 2% | Thermal |
| | 20% | | | |
| F34 | Buprenorphine hexanoate, | NMP, 78% | BHA, 0.2% | Thermal |
| | 20% | | | |
| F35 | Buprenorphine hexanoate, 5% | NMP, 94.5% | VitE, 0.5% | Thermal |
| F36 | Buprenorphine hexanoate, | NMP, 39.5% | VitE, 0.5% | Thermal |
| | 60% | | | |
| F37 | Buprenorphine hexanoate, | NMP, 79.9% | VitC, 0.1% | Thermal |
| | 20% | | | |
| F38 | Buprenorphine hexanoate, | NMP, 78% | TPGS, 2% | Thermal |
| | 20% | | | |
| F39 | Buprenorphine hexanoate, | DMA, 80% | — | Thermal |
| | 20% | | | |
| F40 | Buprenorphine hexanoate, | DMA, 78% | VitE, 2% | Thermal |
| | 20% | | | |

NMP: N-methyl-2-pyrrolidone; DMA: N,N-dimethylacetamide; VitE: vitamin E; VitC: vitamin C; BHA: butylated hydroxyanisole; TPGS: D-α-tocopherol polyethylene glycol 1000 succinate.

### Example 2. Stability Study

For the stability study, Formulations F01 to F27 prepared in Example 1 were stored at 60° C. for 1 day, and Formulation F05, F06, F14, F20, F21, F22, F24, F25, F26, F27 were also tested for storage at 40° C. for 1 month. Further, Formulations F28 to F40 prepared in Example 1 were tested for storage at 25° C. or 40° C. for a given period. It should be noted that the storage conditions at 40° C. or 60° C. used herein are served as accelerated storage conditions.

To measure the amount of impurities in the formulation after storage, each sample was analyzed with high performance liquid chromatography (HPLC) for buprenorphine derivatives, buprenorphine free base, Impurities A to C, and other impurities by the following conditions:

HPLC instrument: Alliance 1260 Infinity; column: Waters Xterra C18, 4.6 mm×250 mm, 5 m; mobile phase A: 0.1% trifluoroacetic acid in water; mobile phase B: methanol; flow rate: 1 mL/min; UV detection (DAD) 210 nm; column temperature: 30° C.; injection volume: 10 μL.

To evaluate the appearance of the formulation after storage, each sample was diluted 3-fold with acetonitrile, and then the color scale of the sample was recorded by using Lovibond EC 2000 Gardner. The Gardner color scale is a one-dimensional scale used to measure the yellowness of transparent liquids. The color score of Gardner 1 is a light yellow, and the color score of Garner 18 is a dark brown.

The results of the stability study of Formulations F28 and F29 were shown in Tables 2 and 3 below. Impurity A1 was found in the formulations of buprenorphine hexanoate by using HPLC.

TABLE 2
The results of stability test to the formulations containing buprenorphine
hexanoate (R₁ = hexanoyl) stored at 25° C. for 1 month to 6 months

| | | | | | Impurity | Total |
|---|---|---|---|---|---|---|
| Formulation | | Storage | | Buprenorphine | A1 | impurities |
| code | API | period | Stabilizer | free base | (%) | (%) |
| F28 | 50% | Initial | 0.2% | ND | ND | ND |
| | Buprenorphine | 1 M | VitE | ND | 0.06 | 0.06 |
| | hexanoate | 2 M | | ND | 0.07 | 0.07 |
| | | 3 M | | ND | 0.12 | 0.22 |
| | | 6 M | | ND | 0.13 | 0.25 |
| F29 | 50% | Initial | — | ND | 0.40 | 0.53 |
| | Buprenorphine | 1 M | | ND | 0.47 | 1.16 |
| | hexanoate | 2 M | | ND | 0.30 | 0.77 |
| | | 3 M | | 0.39 | 0.32 | 1.31 |
| | | 6 M | | 0.31 | 0.27 | 1.09 |

M: month; VitE: vitamin E; ND: not detected.

TABLE 3
The results of stability test to the formulations containing buprenorphine
hexanoate (R₁ = hexanoyl) stored at 40° C. for 1 month to 6 months

| | | | | | Impurity | Total |
|---|---|---|---|---|---|---|
| Formulation | | Storage | | Buprenorphine | A1 | impurities |
| code | API | period | Stabilizer | free base | (%) | (%) |
| F28 | 50% | Initial | 0.2% | ND | ND | ND |
| | Buprenorphine | 1 M | VitE | ND | 0.12 | 0.19 |
| | hexanoate | 2 M | | ND | 0.11 | 0.21 |
| | | 3 M | | ND | 0.14 | 0.23 |
| | | 6 M | | ND | 0.10 | 0.16 |
| F29 | 50% | Initial | — | ND | 0.40 | 0.53 |
| | Buprenorphine | 1 M | | ND | 0.35 | 0.87 |
| | hexanoate | 2 M | | ND | 0.27 | 0.77 |
| | | 3 M | | 0.31 | 0.27 | 1.34 |
| | | 6 M | | 0.31 | 0.29 | 1.12 |

M: month; VitE: vitamin E; ND: not detected.

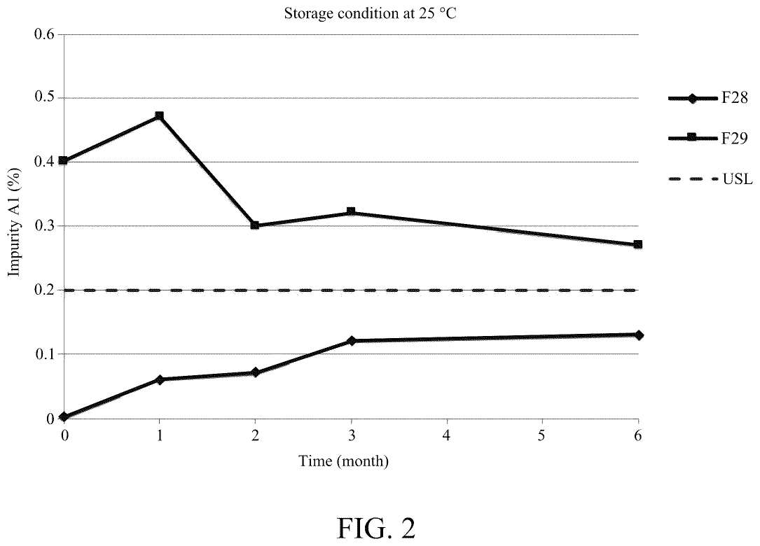

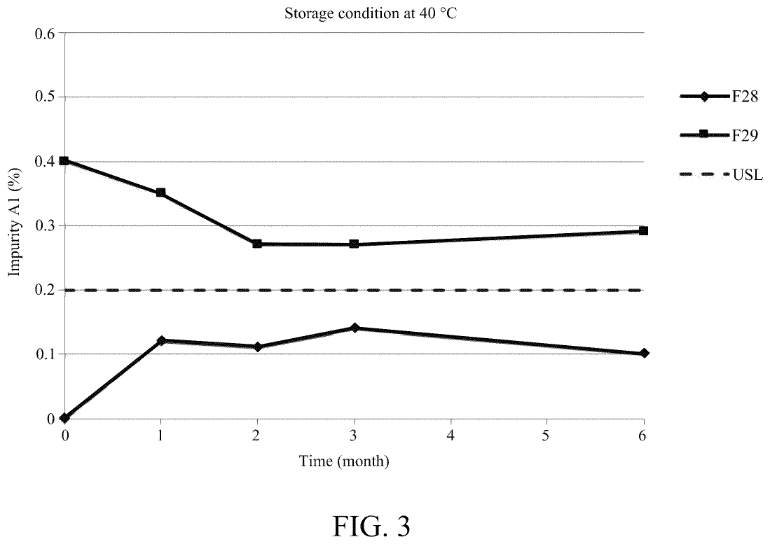

Impurity A1 correlated to impurities having a retention time of 17.5 minutes in the stability test of a buprenorphine hexanoate formulation. The results showed that Formulation F28 added with 0.2% VitE significantly inhibited total impurities and impurity A1 formation under the temperature of 25° C. and 40° C. For example, as shown in FIGS. 2 and 3, by the addition of a stabilizer, less than 0.15% of impurity A1 was produced. It is noted that after 6-month storage at 25° C., as compared to Formulation F29 without a stabilizer, Formulation F28 added with a stabilizer significantly reduced the formation of Impurity A1 by about 51.9% (i.e., 0.13% in Formulation F28, while 0.27% in Formulation F29). Similarly, after 6-month storage at 40° C., as compared to Formulation F29 without a stabilizer, Formulation F28 added with a stabilizer even reduced the formation of Impurity A1 by about 65.5% (i.e., 0.1% in Formulation F28, while 0.29% in Formulation F29).

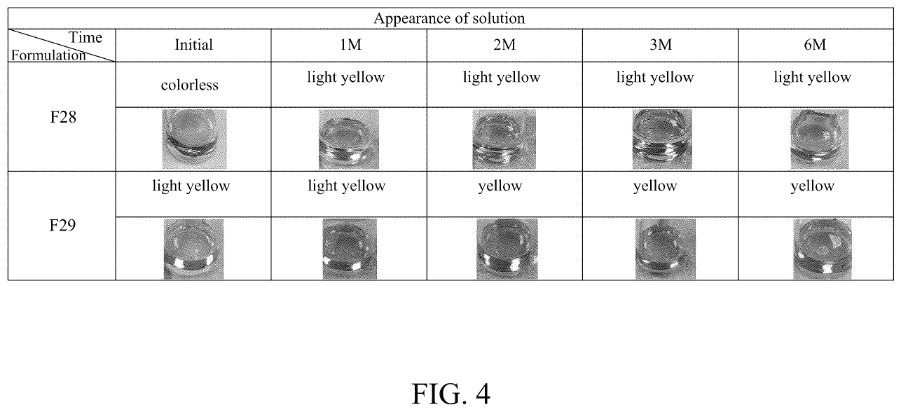

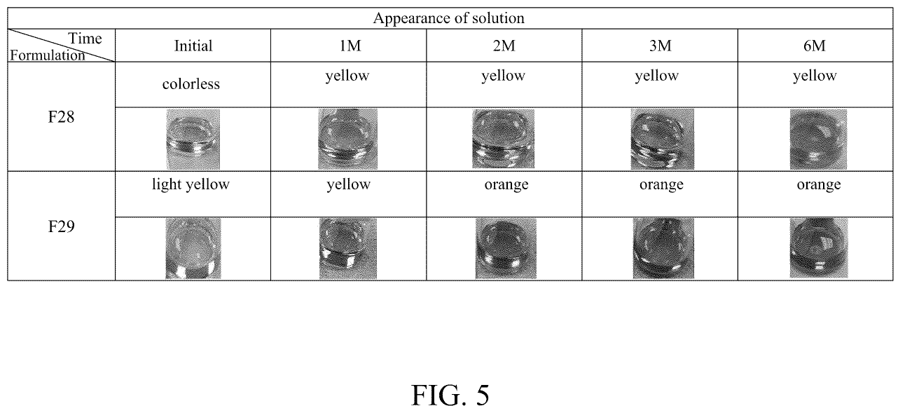

Moreover, the appearance of formulations was shown in FIGS. 4 and 5, illustrating that Formulation F29 showed severe discoloration relative to Formulation 28. Further, the formation profiles of Impurity A1 stored at 25° C. and 40° C. were also shown in FIGS. 2 and 3, respectively, wherein USL represents the upper specification limit, indicating that the impurities each should be less than 0.2% in the new drug product specification according to the ICH guidance Q3B (R2).

These results showed that as compared to Formulation F29 without adding any stabilizers, the addition of 0.2% VitE as the stabilizer in Formulation F28 apparently inhibited the formation of Impurity A1 at 25° C. and 40° C., and the amount of Impurity A1 could be remained lower than USL for at least 6 months. It was also noted that the proportion of Impurity A1 in Formulation F29 was decreased due to the gradual formations of buprenorphine free base and individual unknown impurities within 6 months, while no buprenorphine free base was observed from Formulation F28.

In addition, the results of the stability study of Formulations F30 to F40 were shown in Tables 4 below.

TABLE 4
The results of stability test to the formulations containing buprenorphine decanoate
(R₁ = decanoyl) at 40° C. for a given period of time

| | | | | Impurity | Impurity | Total |
|---|---|---|---|---|---|---|
| Formulation | | Storage | | A2 | C2 | impurities |
| code | API | period | Stabilizer | (%) | (%) | (%) |
| F30 | 20% | Initial | — | 1.00 | ND | 2.56 |
| | Buprenorphine | 14 | | 1.23 | ND | 4.66 |
| | decanoate | days | | | | |
| F31 | 20% | Initial | 0.5% | 0.25 | ND | 0.66 |
| | Buprenorphine | 14 | VitE | 0.48 | ND | 1.03 |
| | decanoate | days | | | | |

VitE: vitamin E; ND: not detected.

TABLE 5
The results of stability test to the formulations containing buprenorphine hexanoate
(R₁ = hexanoyl) at 40° C. for a given period of time

| | | | | Impurity | Impurity | Total |
|---|---|---|---|---|---|---|
| Formulation | | Storage | | A1 | C1 | Impurities |
| code | API | period | Stabilizer | (%) | (%) | (%) |
| F32 | 20% | Initial | 0.1% | 0.16 | ND | 0.16 |
| | Buprenorphine | 14 days | VitE | 0.46 | ND | 0.86 |
| | hexanoate | | | | | |
| F33 | 20% | Initial | 2% | 0.14 | ND | 0.14 |
| | Buprenorphine | 14 days | VitE | 0.23 | ND | 0.23 |
| | hexanoate | | | | | |
| F34 | 20% | Initial | 2% | 0.14 | ND | 0.38 |
| | Buprenorphine | 14 days | BHA | | | |
| | hexanoate | | | 0.46 | ND | 1.36 |
| F35 | 5% | Initial | 0.5% | 0.12 | ND | 0.12 |
| | Buprenorphine | 14 days | VitE | 0.29 | ND | 0.50 |
| | hexanoate | | | | | |
| F36 | 60% | Initial | 0.5% | ND | ND | ND |
| | Buprenorphine | 14 days | VitE | 0.12 | ND | 0.23 |
| | hexanoate | | | | | |
| F37 | 20% | Initial | 0.1% | ND | ND | 0.77 |
| | Buprenorphine | 14 days | VitC | 0.84 | ND | 1.03 |
| | hexanoate | | | | | |
| F38 | 20% | Initial | 2% | 0.26 | ND | 0.26 |
| | Buprenorphine | 14 days | TPGS | 0.57 | ND | 1.10 |
| | hexanoate | | | | | |
| F39 | 20% | Initial | — | 0.55 | ND | 0.78 |
| | Buprenorphine | 14 days | | 0.70 | ND | 1.69 |
| | hexanoate | | | | | |
| F40 | 20% | Initial | 2% | 0.05 | ND | 0.10 |
| | Buprenorphine | 14 days | VitE | 0.08 | ND | 0.08 |
| | hexanoate | | | | | |

VitE: vitamin E; VitC: vitamin C; BHA: butylated hydroxyanisole; TPGS: D-α-tocopherol polyethylene glycol 1000 succinate; ND: not detected.

Impurities A1, A2 and C1 were found in the formulations of buprenorphine derivatives by using HPLC. Impurities A1 and C1 correlated to the impurities having a retention time of 17.5 minutes and 18.9 minutes, respectively, in the stability test of the buprenorphine hexanoate formulation. Impurity A2 correlated to the impurity having a retention time of 30.1 minutes, respectively, in the stability test of the buprenorphine decanoate formulation.

The above results show that the addition of a stabilizer in formulations containing buprenorphine derivatives apparently decreased the formation of total impurities. For example, Table 4 shows that in comparison to Formulation F30 without a stabilizer, Formulation F31 added with 0.5% VitE significantly decreased the formation of total impurities and Impurity A2. Specifically, the non-oxidation impurity (i.e., Impurity A2) of up to 1.23% was generated in Formulation F30 after 14 days, whereas the non-oxidation impurity of less than 0.50% was generated in Formulation F31 after 14 days, with as high as around 61% decrease in Impurity A2.

Further, as shown in Table 5, Formulations F33 and F40 each added with 2% VitE produced relatively low amounts of total impurities (0.23% and 0.08%) after 14 days, and the non-oxidation impurity (i.e., Impurity A1) of less than 0.25% were observed therein.

Also, after 14 days, there was also an apparent decrease in the formation of Impurity A1 and Impurity C1 for the formulations added with BHA or TPGS (i.e., Formulations F34 and F38). Specifically, the total amounts of Impurities A1 and C1 of less than 0.60% was observed in Formulations F34 and F38 after 14 days.

The results of the stability study of Formulations F01 to F27 were shown in Tables 6 to 12 below.

TABLE 6
The results of stability test to the formulations containing buprenorphine
free base (R₁ = H) stored at 60° C. for 1 day

| | | | | | Total |
|---|---|---|---|---|---|
| | | | | Gardner | impu- |
| Formulation | | | Sterili- | color | rities |
| code | API | Stabilizer | zation | scale | (%) |
| F01 | 20% | — | Filtration | 5.2 | 0.66 |
| | Buprenorphine | | | | |
| | free base | | | | |
| F02 | 20% | 0.5% | Filtration | 8.0 | 2.43 |
| | Buprenorphine | VitE | | | |
| | free base | | | | |
| F03 | 20% | — | Thermal | 6.2 | 0.81 |
| | Buprenorphine | | | | |
| | free base | | | | |
| F04 | 20% | 0.5% | Thermal | 7.7 | 1.95 |
| | Buprenorphine | VitE | | | |
| | free base | | | | |

VitE: vitamin E

From the results of Table 6, it was found that the addition of VitE did not significantly decrease the formation of impurities in the formulations containing buprenorphine free base, implying that although VitE is regarded as an antioxidant, it might not inhibit the oxidation of buprenorphine free base.

TABLE 7
The results of stability test to the formulations containing buprenorphine
acetate (R₁ = acetyl) stored at 60° C. for 1 day

| | | | Gardner | Impurity | Impurity | Total |
|---|---|---|---|---|---|---|
| Formulation | | | color | A3 | B3 | impurities |
| code | API | Stabilizer | scale | (%) | (%) | (%) |
| F05 | 20% | — | 4.2 | 0.11 | 0.23 | 0.34 |
| | Buprenorphine | | | | | |
| | acetate | | | | | |
| F06 | 20% | 0.5% | 2.0 | ND | 0.08 | 0.08 |
| | Buprenorphine | VitE | | | | |
| | acetate | | | | | |

VitE: vitamin E; ND: not detected.

TABLE 8
The results of stability test to the formulations containing buprenorphine
hexanoate (R₁ = hexanoyl) stored at 60° C. for 1 day

| | | | | Gardner | Impurity | Impurity | Impurity | Total |
|---|---|---|---|---|---|---|---|---|
| Formulation | | | | color | A1 | B1 | C1 | impurities |
| code | API | Stabilizer | Sterilization | scale | (%) | (%) | (%) | (%) |
| F07 | 20% | — | Filtration | 4.6 | 1.42 | 1.37 | ND | 3.94 |
| | Buprenorphine | | | | | | | |
| | hexanoate | | | | | | | |
| F08 | 20% | 0.01% | Filtration | 1.0 | 0.28 | 0.24 | ND | 0.52 |
| | Buprenorphine | VitE | | | | | | |
| | hexanoate | | | | | | | |
| F09 | 20% | 0.1% | Filtration | 0.8 | 0.29 | 0.16 | ND | 0.55 |
| | Buprenorphine | VitE | | | | | | |
| | hexanoate | | | | | | | |
| F10 | 20% | 0.2% | Filtration | 0.8 | 0.25 | 0.13 | ND | 0.47 |
| | Buprenorphine | VitE | | | | | | |
| | hexanoate | | | | | | | |
| F11 | 20% | 0.5% | Filtration | 0.9 | 0.22 | 0.10 | ND | 0.31 |
| | Buprenorphine | VitE | | | | | | |
| | hexanoate | | | | | | | |
| F12 | 20% | 2% | Filtration | 1.2 | 0.20 | ND | ND | 0.20 |
| | Buprenorphine | VitE | | | | | | |
| | hexanoate | | | | | | | |
| F17 | 20% | 0.5% | Filtration | 1.5 | ND | ND | 0.92 | 0.92 |
| | Buprenorphine | VitC | | | | | | |
| | hexanoate | | | | | | | |
| F18 | 20% | 0.5% | Filtration | 1.4 | 0.29 | 0.24 | ND | 0.76 |
| | Buprenorphine | BHA | | | | | | |
| | hexanoate | | | | | | | |
| F19 | 20% | 0.5% | Filtration | 4.2 | 1.09 | 0.95 | ND | 2.51 |
| | Buprenorphine | TPGS | | | | | | |
| | hexanoate | | | | | | | |
| F15 | 20% | — | Thermal | 5.4 | 0.94 | 1.12 | 0.27 | 3.22 |
| | Buprenorphine | | | | | | | |
| | hexanoate | | | | | | | |
| F16 | 20% | 0.5% | Thermal | 1.1 | 0.42 | 0.18 | ND | 0.91 |
| | Buprenorphine | VitE | | | | | | |
| | hexanoate | | | | | | | |
| F23 | 5% | — | Filtration | 8.1 | 0.47 | 0.42 | 0.35 | 5.59 |
| | Buprenorphine | | | | | | | |
| | hexanoate | | | | | | | |
| F13 | 5% | 0.5% | Filtration | 1.7 | 0.25 | ND | ND | 0.36 |
| | Buprenorphine | VitE | | | | | | |
| | hexanoate | | | | | | | |
| F24 | 50% | — | Filtration | 4.0 | 0.57 | 0.80 | N/A | 1.78 |
| | Buprenorphine | | | | | | | |
| | hexanoate | | | | | | | |
| F22 | 50% | 0.2% | Filtration | 1.8 | 0.11 | ND | N/A | 0.11 |
| | Buprenorphine | VitE | | | | | | |
| | hexanoate | | | | | | | |
| F14 | 50% | 0.5% | Filtration | 1.6 | 0.23 | 0.17 | N/A | 0.40 |
| | Buprenorphine | VitE | | | | | | |
| | hexanoate | | | | | | | |

VitE: vitamin E; VitC: vitamin C; BHA: butylated hydroxyanisole; TPGS: D-α-tocopherol polyethylene glycol 1000 succinate; ND: not detected; N/A: not applicable.

TABLE 9
The results of stability test to the formulations containing buprenorphine
decanoate (R₁ = decanoyl) stored at 60° C. for 1 day

| | | | Gardner | Impurity | Impurity | Total |
|---|---|---|---|---|---|---|
| Formulation | | | color | A2 | B2 | impurities |
| code | API | Stabilizer | scale | (%) | (%) | (%) |
| F20 | 20% | — | 3.7 | 0.96 | 0.91 | 2.68 |
| | Buprenorphine | | | | | |
| | decanoate | | | | | |
| F21 | 20% | 0.5% | 1.6 | 0.26 | ND | 0.57 |
| | Buprenorphine | VitE | | | | |
| | decanoate | | | | | |

VitE: vitamin E; ND: not detected.

Further, Tables 7 to 9 show that the addition of a stabilizer in formulations containing different buprenorphine derivates apparently decreased the formation of total impurities and non-oxidation impurities after storage at 60° C. for 1 day. For example, each of the non-oxidation impurities (i.e., A1 to A3, B1 to B3, and C1) generated in the formulations containing a stabilizer (e.g., VitE and BHA) could be at an amount lower than about 0.5%, and the total impurities could be at an amount lower than about 1%. In contrast, the formulations without any stabilizers (i.e., Formulations F05, F07, F15, F20, F23 and F24) not only exhibited higher color scales, but also generated higher amounts of total impurities of up to about 1.7% to 5.6%.

TABLE 10
The results of stability test to the formulations containing buprenorphine
acetate (R₁ = acetyl) stored at 40° C. for 1 month

| | | | Gardner | Impurity |
|---|---|---|---|---|
| Formulation | | | color | A3 |
| code | API | Stabilizer | scale | (%) |
| F05 | 20% | — | 3.9 | 0.16 |
| | Buprenorphine | | | |
| | acetate | | | |
| F06 | 20% | 0.5% | 1.2 | ND |
| | Buprenorphine | VitE | | |
| | acetate | | | |

VitE: vitamin E; ND: not detected.

TABLE 11
The results of stability test to the formulations containing buprenorphine
hexanoate (R₁ = hexanoyl) stored at 40° C. for 1 month

| | | | Impurity | Impurity | Impurity | Total |
|---|---|---|---|---|---|---|
| Formulation | | | A1 | B1 | C1 | impurities |
| code | API | Stabilizer | (%) | (%) | (%) | (%) |
| F24 | 50% | — | 0.16 | 0.69 | 0.22 | 1.29 |
| | Buprenorphine | | | | | |
| | hexanoate | | | | | |
| F22 | 50% | 0.2% | 0.16 | 0.30 | ND | 0.83 |
| | Buprenorphine | VitE | | | | |
| | hexanoate | | | | | |
| F14 | 50% | 0.5% | 0.15 | 0.28 | ND | 0.78 |
| | Buprenorphine | VitE | | | | |
| | hexanoate | | | | | |

VitE: vitamin E; ND: not detected.

TABLE 12
The results of stability test to the formulations containing buprenorphine
decanoate (R₁ = decanoyl) stored at 40° C. for 1 month

| | | | Gardner | Impurity | Impurity | Total |
|---|---|---|---|---|---|---|
| Formulation | | | color | A2 | B2 | impurities |
| code | API | Stabilizer | scale | (%) | (%) | (%) |
| F20 | 20% | — | 3.2 | 0.49 | 1.05 | 1.83 |
| | Buprenorphine | | | | | |
| | decanoate | | | | | |
| F21 | 20% | 0.5% | 1.1 | 0.22 | 0.41 | 1.46 |
| | Buprenorphine | VitE | | | | |
| | decanoate | | | | | |

VitE: vitamin E

Similarly, Tables 10 to 12 show that after storage at 40° C. for 1 month, the addition of a stabilizer in formulations containing different buprenorphine derivates still has the ability to decrease the formation of total impurities and non-oxidation impurities and prevented discoloration during long-term storage.

From the above results, it was observed that the addition of a stabilizer (e.g., VitE, VitC, and BHA) could effectively inhibit the formation of non-oxidation impurities in the stability test. The stabilizers used herein were generally regarded as antioxidants from prior knowledge. However, the present disclosure proved that the stabilizers unexpectedly decreased impurities also formed by non-oxidation pathways.

While the disclosure has been described with respect to a limited number of embodiments, those skilled in the art, having the benefit of this disclosure, will appreciate that other embodiments can be devised which do not depart from the scope of the present disclosure as disclosed herein. Accordingly, the scope of the present disclosure should be defined only by the attached claims.

1-20. (canceled)

21. A pharmaceutical composition having a shelf life of at least twenty months, comprising:
at least one active pharmaceutical ingredient chosen from buprenorphine, a derivative thereof, or a pharmaceutically acceptable salt thereof;
at least one stabilizer effective to stabilize the at least one active pharmaceutical ingredient during the shelf life;
at least one biocompatible solvent effective to form a mixture in the pharmaceutical composition of the at least one active ingredient and the at least one stabilizer; and
at least one compound of formulae (IIA), (IIB), or (IIC): 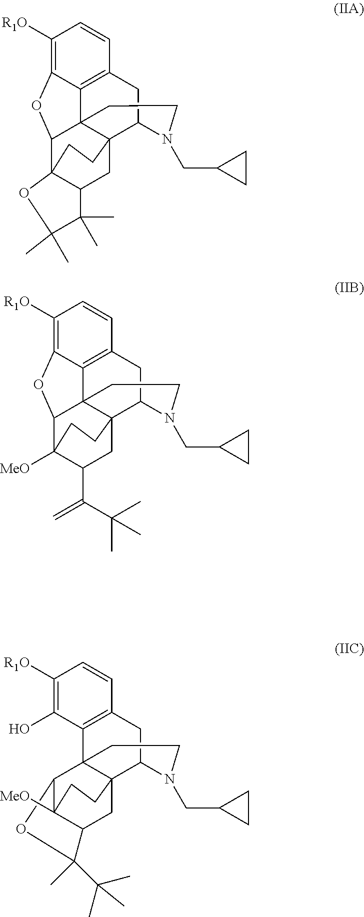
wherein in formulae (IIA), (IIB), and (IIC), R₁ is independently chosen from hydrogen, C₁-C₂₀ alkylcarbonyl groups, or C₆-C₁₈ arylcarbonyl groups; and
wherein the compounds of formulae (IIA), (IIB), and (IIC) are independently present in amounts less than 1% w/w, based on the total weight of the pharmaceutical composition, during the shelf life of the pharmaceutical composition.

22. The pharmaceutical composition according to claim 21, wherein the compounds of formula (IIA), (IIB), and (IIC) are independently present in amounts less than 0.5% w/w, based on the total weight of the pharmaceutical composition, during the shelf life of the pharmaceutical composition.

23. The pharmaceutical composition according to claim 21, wherein the shelf life is at least twenty months at ambient temperature or a temperature below ambient temperature.

24. The pharmaceutical composition according to claim 21, wherein the at least one active pharmaceutical ingredient is chosen from compounds of formula (I): 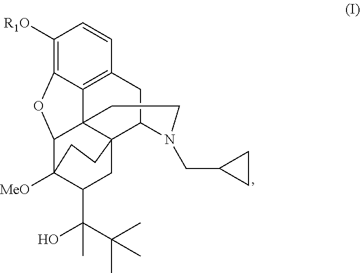
wherein R₁ is chosen from hydrogen, C₁-C₂₀ alkylcarbonyl groups, or C₆-C₁₈ arylcarbonyl groups.

25. The pharmaceutical composition according to claim 21, wherein the at least one active pharmaceutical ingredient is present in an amount ranging from 5% w/w to 80% w/w, based on the total weight of the pharmaceutical composition.

26. The pharmaceutical composition according to claim 21, wherein the at least one stabilizer is present in an amount ranging from 0.01% w/w to 10% w/w, based on the total weight of the pharmaceutical composition.

27. The pharmaceutical composition according to claim 21, wherein the at least one stabilizer is chosen from vitamin E, a derivative of vitamin E, ascorbic acid, butylated hydroxyanisole, and combinations of two or more thereof.

28. The pharmaceutical composition according to claim 27, wherein the vitamin E or derivative of vitamin E are chosen from α-tocopherol, β-tocopherol, γ-tocopherol, 5-tocopherol, α-tocotrienol, β-tocotrienol, γ-tocotrienol, 5-tocotrienol, D-α-tocopherol polyethylene glycol 1000 succinate, and combinations of two or more thereof.

29. The pharmaceutical composition according to claim 21, wherein the at least one biocompatible solvent is chosen from N-methyl-2-pyrrolidone, polyethylene glycol 400, polyethylene glycol 4000, ethyl acetate, ethanol, butanol, 2-butanol, isobutanol, isopropanol, glycerin, benzyl benzoate, dimethyl sulfoxide, N,N-dimethylacetamide, propylene glycol, dimethyl glycol, benzyl alcohol, and combinations of two or more thereof.

30. The pharmaceutical composition according to claim 21, wherein the composition has a color score of less than Gardner 4.

31. A method of preparing the pharmaceutical composition according to claim 21, the method comprising adding at least one active pharmaceutical ingredient and at least one stabilizer in at least one biocompatible solvent to form the pharmaceutical composition, wherein the at least one active pharmaceutical ingredient is chosen from buprenorphine, a derivative thereof, and a pharmaceutically acceptable salt thereof.

32. The method according to claim 31, further comprising stirring the pharmaceutical composition to form a homogeneous solution.

33. The method according to claim 32, wherein the stirring is performed with heating.

34. The method according to claim 32, further comprising filtering the homogeneous solution to obtain a filtered homogeneous solution.

35. The method according to claim 32, further comprising sterilizing the homogeneous solution to obtain a sterilized pharmaceutical composition.

36. The method according to claim 35, wherein the sterilizing is chosen from thermal sterilization, filtration sterilization, irradiation sterilization, and combinations or two or more thereof.

37. The method according to claim 31, wherein the compounds of formulae (IIA), (IIB), and (IIC) are formed by degradation of the at least one active pharmaceutical ingredient.

38. A method for treating opioid addiction, pain, and/or depression in a human subject in need thereof, comprising administering to the human subject a therapeutically effective amount of the pharmaceutical composition according to claim 21.

39. The method according to claim 38, wherein the pharmaceutical composition is administered to the human subject three times per day, two times per day, once per day, once every two days, once every three days, once every four days, once every five days, once every six days, once per week, two times per week, three times per week, four times per week, five times per week, six times per week, once every two weeks, once every three weeks, once every four weeks, once every five weeks, once every six weeks, once every eight weeks, once every ten weeks, once every twelve weeks, once every four months, once every five months, or once every six months.

40. The method according to claim 38, wherein the pharmaceutical composition is administered to the human subject via subcutaneous administration, intravenous administration, intramuscular administration, intradermal administration, transdermal administration, sublingual administration, topical administration, intraperitoneal administration, intraarticular administration, transmucosal administration, intraorgan administration, intraosseous administration, or oral administration.
